(19) United States
(12) Patent Application Publication
(10) Pub. No.: US 2012/0135522 A1
(43) Pub. Date: May 31, 2012

(54) METHODS OF MODULATING CELL REGULATION BY INHIBITING P53

(76) Inventor: John S. Kovach, East Setauket, NY (US)

(21) Appl. No.: 13/378,623

(22) PCT Filed: Feb. 1, 2010
(86) PCT No.: PCT/US2010/000279

§ 371 Date: Feb. 17, 2012

Related U.S. Application Data
(60) Provisional application No. 61/269,101, filed on Jun. 18, 2009.

(30) Foreign Application Priority Data
Jul. 16, 2009 (US) 2009/004108

(51) Int. Cl. C12N 5/02

(52) U.S. Cl. 435/375; 435/377

(57) ABSTRACT

Disclosed herein are methods of inhibiting the function of p53 in a cell by contacting the cell with an effective amount of a PP2A inhibitor. Also disclosed herein are processes for producing an induced pluripotent stem (iPS) cell by contacting a somatic cell expressing at least one gene that encodes a reprogramming factor with an amount of a PP2A inhibitor effective to produce the iPS cell; reversibly inhibiting p53 function during production of an iPS cell by contacting a somatic cell with an amount of a PP2A inhibitor effective to reversibly inhibit p53 function; increasing the likelihood of producing an (iPS) cell.

This application claims priority of (i) PCT International Application No. PCT/US2009/004108, filed Jul. 16, 2009 and (ii) U.S. Provisional Application No. 61/269,101, filed Jun. 18, 2009, the contents of each of which in its entirety is hereby incorporated by reference.

Throughout this application, certain publications are referenced. Full citations for these publications may be found immediately preceding the claims. The disclosures of these publications in their entireties are hereby incorporated by reference into this application in order to describe more fully the state-of-the art to which this invention relates.

### BACKGROUND OF THE INVENTION

There is an intense worldwide research effort to develop technology that reliably induces development of functional genetically intact pluripotent stem cells (iPS) from somatic cells. It has been shown that inserting as few as 3 or 4 genes into an adult mammalian cell is sufficient to convert that cell into an embryo-like state believed to be capable of developing into any tissue of the body (Science Feb. 1, 2008). A major difficulty in developing iPS cells is the inefficiency of the conversion process in which generally fewer than 1 percent of adult cells are successfully re-programmed into iPS cells.

### SUMMARY OF THE INVENTION

Provided herein is a method of inhibiting the function of p53 in a cell comprising contacting the cell with an effective amount of a PP2A inhibitor, wherein the PP2A inhibitor is a compound having the structure:

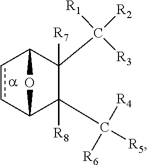

wherein
bond α is present or absent;
R₁ and R₂ is each independently H, O⁻ or OR₉,
   where R₉ is H, alkyl, substituted alkyl, alkenyl, alkynyl or aryl,
or R₁ and R₂ together are ═O;
R₃ and R₄ are each different, and each is OH, O⁻, OR₉, OR₁₀, O(CH₂)₁₋₆R₉, SH, S⁻, SR₉,

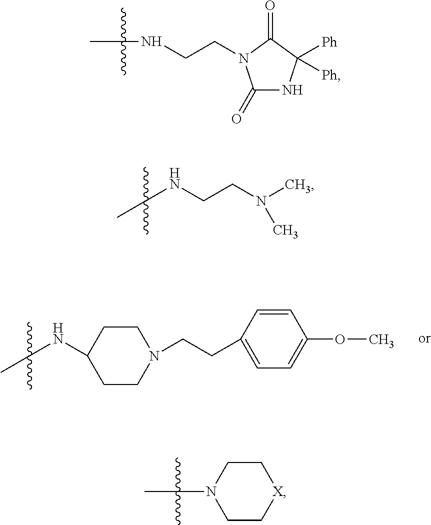

where X is O, S, NR₁₁, or N⁺R₁₁R₁₁,
   where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

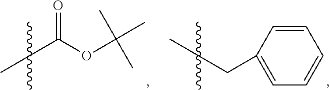

—CH₂CN, —CH₂CO₂R₁₂, —CH₂COR₁₂, —NHR₁₂ or —NH⁺(R₁₂)₂, where each R₁₂ is independently alkyl, alkenyl or alkynyl, each of which is substituted or unsubstituted, or H;
R₁₀ is substituted alkyl, substituted alkenyl, substituted alkynyl, or substituted aryl;
R₅ and R₆ is each independently H, OH, or R₅ and R₆ taken together are ═O; and
R₇ and R₈ is each independently H, F, Cl, Br, SO₂Ph, CO₂CH₃, or SR_{A},
   where R₁₃ is H, aryl or a substituted or unsubstituted alkyl, alkenyl or alkynyl,
or a salt, enantiomer or zwitterion of the compound, so as to thereby inhibit the function of p53 in the cell.

Also provided is a process for producing an induced pluripotent stem (iPS) cell comprising contacting a somatic cell expressing at least one gene that encodes a reprogramming factor with an amount of a PP2A inhibitor effective to produce the iPS cell.

Further provided is a process for reversibly inhibiting p53 function during production of an induced pluripotent stem (iPS) cell from a somatic cell expressing at least one gene that encodes a reprogramming factor comprising contacting the somatic cell with an amount of a PP2A inhibitor effective to reversibly inhibit p53 function.

Disclosed herein is also a process for increasing the likelihood of producing an induced pluripotent stem (iPS) cell comprising contacting a somatic cell expressing at least one gene that encodes a reprogramming factor with an amount of a PP2A inhibitor effective to increase the likelihood of producing an iPS.

Disclosed herein is also a process for increasing the production efficiency of induced pluripotent stem (iPS) cells, wherein the process for production of the iPS cells comprises transforming a population of somatic cells with at least one gene that encodes a reprogramming factor, the process comprising contacting the population of somatic cells with an amount of a PP2A inhibitor effective to increase the efficiency of the production of iPS cells.

Disclosed herein is also a process of producing an induced pluripotent stem cell (iPS) comprising transforming a somatic cell to express at least one gene that encodes a reprogramming factor such that the somatic becomes an iPS, wherein the improvement comprises contacting the somatic cell with an amount of a PP2A inhibitor effective to transiently reduce the function of p53 in the cell.

### BRIEF DESCRIPTION OF THE FIGURES

FIG. 1: Inhibition of PP2A and PP1 Activition

Inhibition of PP2A and PP1 activity in lysates of human glioblastoma cell line U87 by Compound 102 (mean and s.d.; n=3) (Ser/thr Phosphatase Assay Kit 1, Milliport, Billerica, Mass.).

FIG. 2: Inhibition of PP2A Activity in U87 Subcutaneous (sc) Xenografts and in Normal Brain Tissue.

Inhibition of PP2A activity in U87 subcutaneous (sc) xenografts is shown in the left column and in normal brain issues in the right column of SCID mice at different times after i.p. injection of 1.5 mg/kg compound 102 (one mouse per point; mean of 3 lysates; 1 s.d.).

FIG. 3: Cellular and Molecular Changes in U87 Cells Induced by Compound 102.

Cellular and molecular changes in U87 cells induced by compound 102. Western blot of U87 lysates: pAkt-1, total Akt-1, and β-actin (left panel) and TCTP, pPlk (Tre-210), total Plk, and β-actin (right panel), after 24 hour exposure to 2.5 uM compound 102.

FIG. 4: p53, pMDM2 and β-Actin After Exposure to Compound 102.

P53(ser-15), pMDM2 (ser-166), and β-actin after 24 hour exposure to 2.5 uM compound 102.

FIG. 5: Western Blots of p-Akt-1, p53, pMDM2 and β-Actin in Lysates of U87 Cells.

Western blots of p-Akt-1, p53, pMDM2, and beta actin in lysates of U87 cells 24 hours after exposure to compound 102 at 2.5 uM, Temozolomide (TMZ) at 25 uM, and compound 102 plus TMZ.

FIG. 6: Western Blots of p-Akt-1, p53, and β-Actin in Lysates of U373 Cells.

Western blots of p-Akt-1, p53, and beta actin in lysates of U373 cells 24 hours after exposure to okadaic acid (2 nM), TMZ at 25 uM, and to both drugs at the same concentrations.

### DETAILED DESCRIPTION OF THE INVENTION

The invention provides a method of inhibiting the function of p53 in a cell comprising contacting the cell with an effective amount of a PP2A inhibitor, wherein the PP2A inhibitor is a compound having the structure:

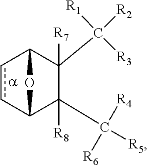

wherein
bond α is present or absent;
R₁ and R₂ is each independently H, O⁻ or OR₉,
   where R₉ is H, alkyl, substituted alkyl, alkenyl, alkynyl or aryl,
or R₁ and R₂ together are ═O;
R₃ and R₄ are each different, and each is OH, O⁻, OR₉, OR₁₀, O(CH₂)₁₋₆R₉, SH, S⁻, SR₉,

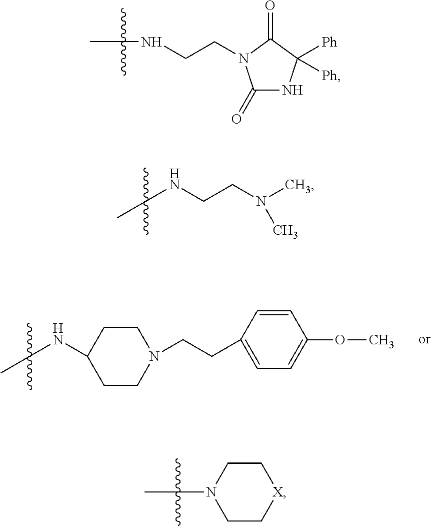

where X is O, S, NR₁₁, or N⁺R₁₁R₁₁,
   where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

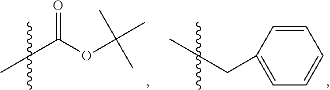

—CH₂CN, —CH₂CO₂R₁₂, —CH₂COR₁₂, —NHR₁₂ or —NH⁺(R₁₂)₂, where each R₁₂ is independently alkyl, alkenyl or alkynyl, each of which is substituted or unsubstituted, or H;
R₁₀ is substituted alkyl, substituted alkenyl, substituted alkynyl, or substituted aryl;
R₅ and R₆ is each independently H, OH, or R₅ and R₆ taken together are ═O; and
R₇ and R₈ is each independently H, F, Cl, Br, SO₂Ph, CO₂CH₃, or SR₁₃,
   where R₁₃ is H, aryl or a substituted or unsubstituted alkyl, alkenyl or alkynyl,
      or a salt, enantiomer or zwitterion of the compound, so as to thereby inhibit the function of p53 in the cell.

In one embodiment of the above method, the compound has the structure

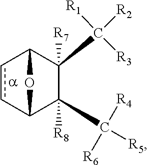

or a salt, enantiomer or zwitterion of the compound.

In another embodiment of the above method, the compound has the structure

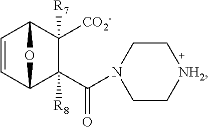

or a salt, enantiomer or zwitterion of the compound.

In a further emdoiment of the above method, R₄ is

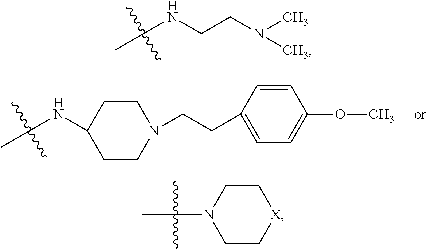

where X is O, NR₁₁, N+R₁₁R₁₁
   where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

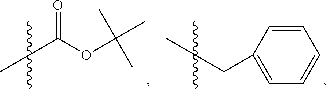

—CH₂CN, —CH₂CO₂R₁₂, —CH₂COR₁₂, —NHR₁₂ or —NH⁺(R₁₂)₂, where R₁₂ is H or alkyl,
   or a salt, enantiomer or zwitterion of the compound.

In a further embodiment, the compound is compound 104, compound 104E, compound 106, or compound 106E; or a salt, enantiomer or zwitterion of the compound.

In another embodiment of the above method, the compound has the structure

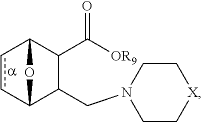

wherein bond α is present or absent; R₉ is present or absent and when present is H, C₁-C₁₀ alkyl, C₂-C₁₀ alkenyl or phenyl; and X is O, S, NR₁₁ or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

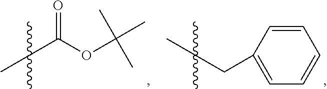

—CH₂CO₂R₁₂, —CH₂COR₁₂, —CH₂CN, or —CH₂CH₂R₁₆, where R₁₂ is H or alkyl, and where R₁₆ is any substituent that is a precursor to an aziridinyl intermediate,
   or a salt, zwitterion or enantiomer of the compound.

In one embodiment of the above method, the compound has the structure

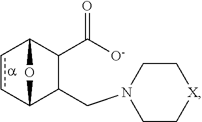

wherein,
bond α is present or absent;
X is O, S, NR₁₁ or N⁺R₁₁R₁₁,
   where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

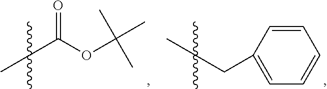

—CH₂CO₂R₁₂, —CH₂COR₁₂, —CH₂CN, or —CH₂CH₂R₁₆, where R₁₂ is H or alkyl, and where R₁₆ is any substitutent that is a aziridinyl intermediate,
   or a salt, zwitterion or enantiomer of a compound.

In another embodiment of the above method, the compound has the structure

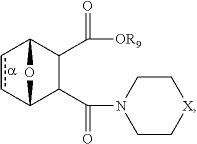

wherein
   bond α is present or absent;
   R₉ is present or absent and when present is H, alkyl, alkenyl, alkynyl or phenyl; and
   X is O, NR₁₁, or N⁺R₁₁R₁₁,
      where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

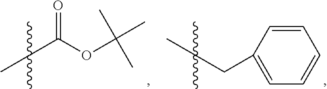

—CH₂CN, —CH₂CO₂R₁₂, or —CH₂COR₁₂, where R₁₂ is H or alkyl,
or a salt, zwitterion, or enantiomer of the compound.

In another embodiment, the compound has the structure

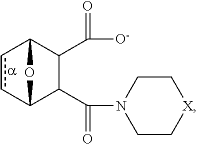

wherein
bond α is present or absent;
X is O or NH⁺R₁₁,
   where R₁₁ is H, alkyl, substituted C₂-C₁₂ alkyl, alkenyl, substituted C₄-C₁₂ alkenyl, alkynyl, substituted alkynyl, aryl, substituted aryl where the substituent is other than chloro,

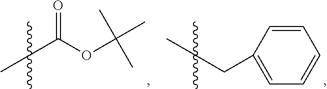

—CH₂CN, —CH₂CO₂R₁₂, or —CH₂COR₁₂, where R₁₂ is H or alkyl,
   or a salt, enantiomer or zwitterion of the compound.

In one embodiment of the above compound, bond α is present. On another embodiment of the above compound, bond α is absent. In a further embodiment, the compound has the structure of compound 100, compound 102, compound 101, compound 103, compound 105, or compound 107; or a salt, entantiomer or zwitterions of the compound. In another embodiment, the compound has the structure of compound 100E, compound 102E, compound 101E, compound 103E, compound 105E, or compound 107E; or a salt, entantiomer or zwitterions of the compound.

In a further embodiment of the above method, the compound has the structure

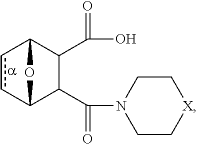

wherein
bond α is present or absent; X is NH⁺R₁₁,
   where R¹¹ is present or absent and when present is alkyl, substituted C₂-C₁₂ alkyl, alkenyl, substituted C₄-C₁₂ alkenyl,

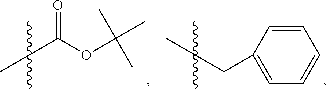

—CH₂CN, —CH₂CO₂R₁₂, or —CH₂COR₁₂, where R₁₂ is H or alkyl,
or a salt, enantiomer or zwitterion of the compound

In a further embodiment of the above method, the compound is compound 108 or a salt enationmer or zwitterions of the compound.

In one embodiment of the above method,
R₃ is OR₁₀ or O(CH₂)₁₋₆R₉,
   where R₉ is aryl or substituted ethyl;
   where R₁₀ is substituted phenyl, wherein the substituent is in the para position;
R₄ is

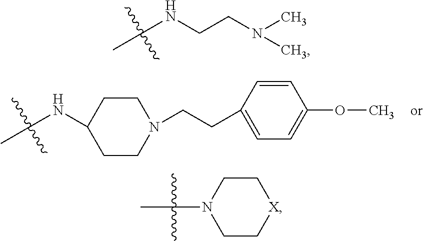

where X is O, S, NR₁₁, or N⁺R₁₁R₁₁,
   where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro,

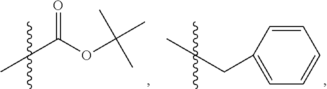

—CH₂CN, —CH₂CO₂R₁₂, —CH₂COR₁₂, —NHR₁₂ or —NH⁺(R₁₂)₂, where R₁₂ is alkyl, alkenyl or alkynyl, each of which is substituted or unsubstituted, or H;
or where R₃ is OH and R₄ is

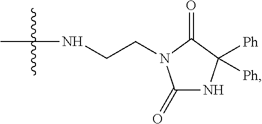

or a salt, enantiomer or zwitterion of the compound.

In a further embodiment of the above method, the compound has the structure

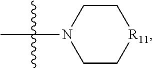

where R₁₁ is alkyl or hydroxylalkyl
or R₄ is

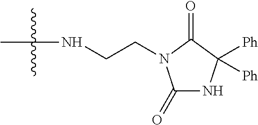

when R₃ is OH,
   or a salt, enantiomer or zwitterion of the compound.

In another embodiment of the above method, the compound has the structure of compound 109, compound 110, compound 112, compound 113, or compound 114; or a salt, enantiomer or zwitterion of the compound. In a further embodiment of the above method, the compound has the structure of compound 109E, compound 110E, compound 112E, compound 113E or compound 114E; or a salt enatniomer or zwitterion of the compound.

In a further embodiment, the compound 108E or compound III; or a salt enatniomer or zwitterion of the compound.

This invention further provides a process for producing an induced pluripotent stem (iPS) cell comprising contacting a somatic cell expressing at least one gene that encodes a reprogramming factor with an amount of a PP2A inhibitor effective to produce the iPS cell.

Also provided is a process for reversibly inhibiting p53 function during production of an induced pluripotent stem (iPS) cell from a somatic cell expressing at least one gene that encodes a reprogramming factor comprising contacting the somatic cell with an amount of a PP2A inhibitor effective to reversibly inhibit p53 function.

This invention further provides a process for increasing the likelihood of producing an induced pluripotent stem (iPS) cell comprising contacting a somatic cell expressing at least one gene that encodes a reprogramming factor with an amount of a PP2A inhibitor effective increa the likelihood of producing an iPS.

This invention also provides a process for increasing the production efficiency of induced pluripotent stem (iPS) cells, wherein the process for production of the iPS cells comprises transforming a population of somatic cells with at least one gene that encodes a reprogramming factor, the process comprising contacting the population of somatic cells with an amount of a PP2A inhibitor effective to increase the efficiency of the production of iPS cells.

This invention further provides a process of producing an induced pluripotent stem cell (iPS) comprising transforming a somatic cell to express at least one gene that encodes a reprogramming factor such that the somatic becomes an iPS, wherein the improvement comprises contacting the somatic cell with an amount of a PP2A inhibitor effective to transiently reduce the function of p53 in the cell.

In one embodiment of any of the above processes, the PP2A inhibitor is a compound having the structure:

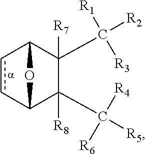

wherein
bond α is present or absent;
R₁ and R₂ is each independently H, O⁻ or OR₉,
   where R₉ is H, alkyl, substituted alkyl, alkenyl, alkynyl or aryl,
or R₁ and R₂ together are ═O;
R₃ and R₄ are each different, and each is OH, O⁻, OR₉, OR₁₀, O(CH₂)₁₋₆R₉, SH, S⁻, SR₉,

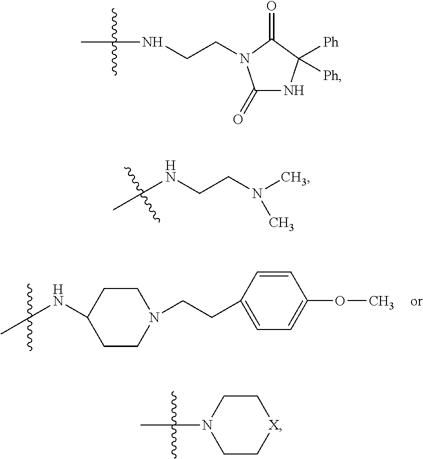

where X is O, S, NR₁₁, or N⁺R₁₁R₁₁,
   where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

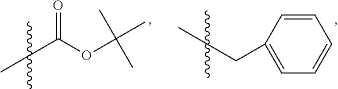

—CH₂CN, —CH₂CO₂R₁₂, —CH₂COR₁₂, —NHR₁₂ or —NH⁺(R₁₂)₂, where each R₁₂ is independently alkyl, alkenyl or alkynyl, each of which is substituted or unsubstituted, or H;
R₁₀ is substituted alkyl, substituted alkenyl, substituted alkynyl, or substituted aryl;
R₅ and R₆ is each independently H, OH, or R₅ and R₆ taken together are ═O; and
R₇ and R₈ is each independently H, F, Cl, Br, SO₂Ph, CO₂CH₃, or SR₁₃,
   where R₁₃ is H, aryl or a substituted or unsubstituted alkyl, alkenyl or alkynyl,
or a salt, enantiomer or zwitterion of the compound.

In one embodiment of the above processes, the compound has the structure

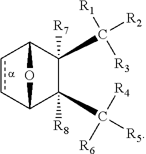

In a further embodiment of the above processes, the compound has the structure

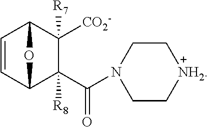

In a further embodiment of the above processes, R₄ is

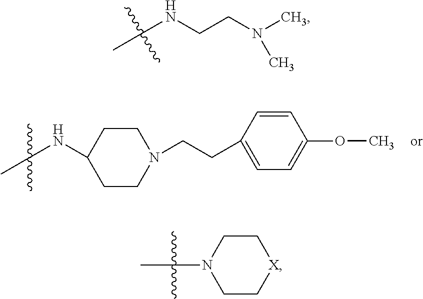

where X is O, NR₁₁, N⁺R₁₁R₁₁
   where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O,

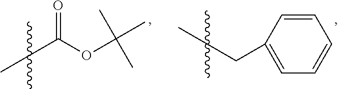

—CH₂CN, —CH₂CO₂R₁₂, —CH₂COR₁₂, —NHR₁₂ or —NH⁺(R₁₂)₂, where R₁₂ is H or alkyl.

In another embodiment of the above processes, the compound is In a further embodiment, the compound is compound 104, compound 104E, compound 106, or compound 106E; or a salt, enantiomer or zwitterion of the compound.

In another embodiment of the above processes, the compound has the structure

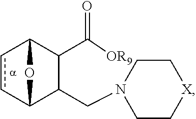

wherein bond α is present or absent; R₉ is present or absent and when present is H, C₁-C₁₀ alkyl, C₂-C₁₀ alkenyl or phenyl; and X is O, S, NR₁₁ or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

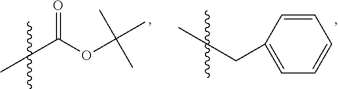

—CH₂CO₂R₁₂, —CH₂COR₁₂, —CH₂CN, or —CH₂CH₂R₁₆, where R₁₂ is H or alkyl, and where R₁₆ is any substituent that is a precursor to an aziridinyl intermediate,
   or a salt, zwitterion or enantiomer of the compound.

In one embodiment of the above processes, the compound has the

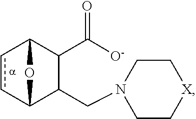

wherein,
bond α is present or absent;
X is O, S, NR₁₁ or N⁺R₁₁R₁₁,
   where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

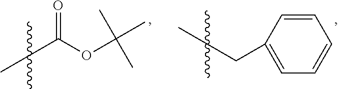

—CH₂CO₂R₁₂, —CH₂COR₁₂, —CH₂CN, or —CH₂CH₂R₁₆, where R₁₂ is H or alkyl, and where R₁₆ is any substitutent that is a aziridinyl intermediate,
   or a salt, zwitterion or enantiomer of a compound.

In another embodiment of the above processes, the compound has the structure

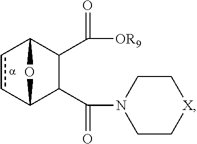

wherein
   bond α is present or absent;
   R₉ is present or absent and when present is H, alkyl, alkenyl, alkynyl or phenyl; and
   X is O, NR₁₁, or N⁺R₁₁R₁₁,
      where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro,

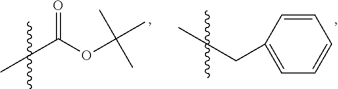

—CH₂CN, —CH₂CO₂R₁₂, or —CH₂COR₁₂, where R₁₂ is H or alkyl,
or a salt, zwitterion, or enantiomer of the compound.

In another embodiment, the compound has the structure

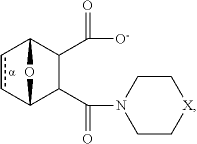

wherein
bond α is present or absent;
X is O or NH⁺R₁₁,
   where R₁₁ is H, alkyl, substituted C₂-C₁₂ alkyl, alkenyl, substituted C₄-C₁₂ alkenyl, alkynyl, substituted alkynyl, aryl, substituted aryl where the substituent is other than chloro,

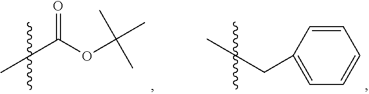

—CH₂CN, —CH₂CO₂R₁₂, or —CH₂COR₁₂, where R₁₂ is H or alkyl,
   or a salt, enantiomer or zwitterion of the compound.

In one embodiment of the above compound, bond α is present. On another embodiment of the above compound, bond α is absent. In a further embodiment, the compound has the structure of compound 100, compound 102, compound 101, compound 103, compound 105, or compound 107; or a salt, entantiomer or zwitterions of the compound. In another embodiment, the compound has the structure of compound 100E, compound 102E, compound 101E, compound 103E, compound 105E, or compound 107E; or a salt, entantiomer or zwitterions of the compound.

In a further embodiment of the above processes, the compound has the structure

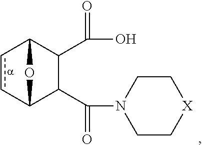

wherein
bond α is present or absent; X is NH⁺R₁₁,
   where R₁₁ is present or absent and when present R₁₁ is alkyl, substituted C₂-C₁₂ alkyl, alkenyl, substituted C₄-C₁₂ alkenyl,

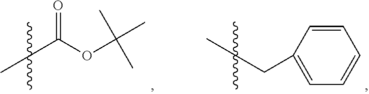

—CH₂CN, —CH₂CO₂R₁₂, or —CH₂COR₁₂, where R₁₂ is H or alkyl,
or a salt, enantiomer or zwitterion of the compound

In a further embodiment of the above processes, the compound is compound 108 or a salt enationmer or zwitterions of the compound.

In one embodiment of the above processes,
R₃ is OR₁₀ or O(CH₂)₁₋₆R₉,
   where R₉ is aryl or substituted ethyl;
   where R₁₀ is substituted phenyl, wherein the substituent is in the para position;
R₄ is

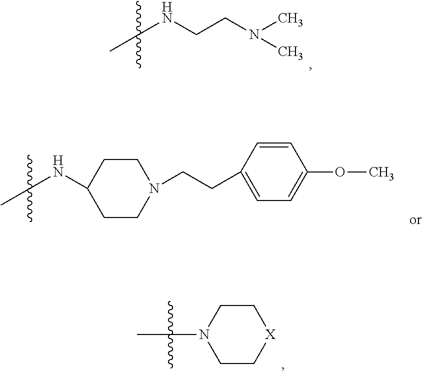

where X is O, S, NR₁₁, or N⁺R₁₁R₁₁,
   where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro,

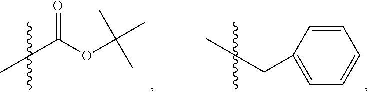

—CH₂CN, —CH₂CO₂R₁₂, —CH₂COR₁₂, —NHR₁₂ or —NH⁺(R₁₂)₂, where R₁₂ is alkyl, alkenyl or alkynyl, each of which is substituted or unsubstituted, or H;
or where R₃ is OH and R₄ is

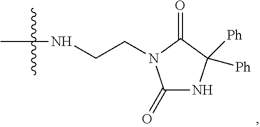

or a salt, enantiomer or zwitterion of the compound.

In a further embodiment of the above processes, the compound has the structure

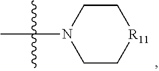

where R₁₁ is alkyl or hydroxylalkyl
or R₄ is

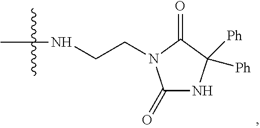

when R₃ is OH,
   or a salt, enantiomer or zwitterion of the compound.

In another embodiment of the above processes, the compound has the structure of compound 109, compound 110, compound 112, compound 113, or compound 114; or a salt, enantiomer or zwitterion of the compound. In a further embodiment of the above processes, the compound has the structure of compound 109E, compound 110E, compound 112E, compound 113E or compound 114E; or a salt enatniomer or zwitterion of the compound.

In a further embodiment, the compound 108E or compound III; or a salt enatniomer or zwitterion of the compound.

### DEFINITIONS

Certain embodiments of the disclosed compounds can contain a basic functional group, such as amino or alkylamino, and are thus capable of forming pharmaceutically acceptable salts with pharmaceutically acceptable acids, or contain an acidic functional group and are thus capable of forming pharmaceutically acceptable salts with bases. The instant compounds therefore may be in a salt form. As used herein, a “salt” is a salt of the instant compounds which has been modified by making acid or base salts of the compounds. The salt may be pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. For a description of possible salts, see, e.g., Berge et al. (1977) “Pharmaceutical Salts”, J. Pharm. Sci. 66:1-19, the contents of which are hereby incorporated by reference.

As used herein, “induced pluripotent stem (iPS) cell” are pluripotent stem cells which are derived from somatic cells by forced expression of genes encoding reprogramming factors, which include, for example, c-Myc, Klf4, Sox2, Oct4(POU5F1), 1 in28 and Nanog. Processes for producing iPS cells have been described, for example, in Takahashi K and Yamanaka S., 2006; Okita K., et al, 2007; Wernig, M. et al, 2007; U.S. Patent Application Publication Nos. US 2006/0205075; US 2009/0047263; US 2009/0227032; and US 2009/0246875; the contents of each of which are hereby incorporated by reference.

As used herein, “reprogramming factor” are genes, such as transcription factors, which can be used for the production of iPS cells. Reprogramming factors include, but are not limited to, c-Myc, Klf4, Sox2, Oct4(POU5F1), 1 in28 and Nanong.

As used herein, increasing the production efficiency of iPS cells is achieved when the amount of iPS cells produced from a population of somatic cells which have been contacted with a PP2A inhibitor is greater than the amount of iPS cells which have not been contacted with a PP2A inhibitor but which have been otherwise been produced by the same process. For example, the increase in production efficiency could be represented by a greater than 5%, greater than 10%, greater than 15%, greater than 20% increase in the amount of iPS cells so produced.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, and so on. An embodiment can be C₁-C₁₂ alkyl. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2, . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2, . . . , n−1 or n carbons. For example, “C₂-C₆ alkynyl” means an alkynyl radical having 2 or 3 carbon atoms, and 1 carbon-carbon triple bond, or having 4 or carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl.

As used herein, “aryl” is intended to mean any stable monocyclic or bicyclic carbon ring of up to 10 atoms in each ring, wherein at least one ring is aromatic. Examples of such aryl elements include phenyl, naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring. The substituted aryls included in this invention include substitution at any suitable position with amines, substituted amines, alkylamines, hydroxys and alkylhydroxys, wherein the “alkyl” portion of the alkylamines and alkylhydroxys is a C₂-Cₙ alkyl as defined hereinabove. The substituted amines may be substituted with alkyl, alkenyl, alkynl, or aryl groups as hereinabove defined.

The alkyl, alkenyl, alkynyl, and aryl substituents may be unsubstituted or unsubstituted, unless specifically defined otherwise. For example, a (C₁-C₆)alkyl may be substituted with one or more substituents selected from OH, oxo, halogen, alkoxy, dialkylamino, or heterocyclyl, such as morpholinyl, piperidinyl, and so on.

In the compounds of the present invention, alkyl, alkenyl, and alkynyl groups can be further substituted by replacing one or more hydrogen atoms by non-hydrogen groups described herein to the extent possible. These include, but are not limited to, halo, hydroxy, mercapto, amino, carboxy, cyano and carbamoyl.

The term “substituted” as used herein means that a given structure has a substituent which can be an alkyl, alkenyl, or aryl group as defined above. The term shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

As used herein, “zwitterion” means a compound that is electrically neutral but carries formal positive and negative charges on different atoms. Zwitterions are polar, have high solubility in water and have poor solubility in most organic solvents.

The compounds disclosed herein may also form zwitterions. For example, a compound having the structure

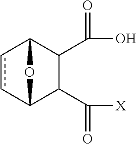

may also for the following zwitterionic structure

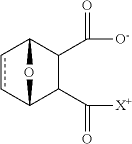

where X is as defined throughout the disclosures herein.

Compounds 100-114 and 100E-110E and 112E-114E, as described herein, were obtained from Lixte Biotechnology, Inc. 248 Route 25A, No. 2, East Setauket, N.Y. The structure of compounds 100-114 and 100E-110E and 112E-114E are listed in Table 1. Processes for making these compounds can be found in PCT International Application Publication WO 2008/097561, the contents of which are hereby incorporated by reference.

TABLE 1
Compounds 100-114; 100E-110E and 112E-114E

| | |
|---|---|
| Compound 100 | 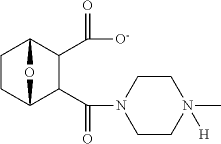 |
| Compound 100E | 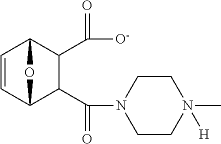 |
| Compound 101 | 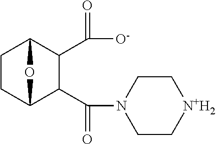 |
| Compound 101E | 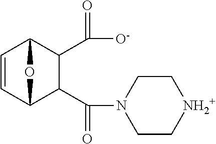 |
| Compound 102 | 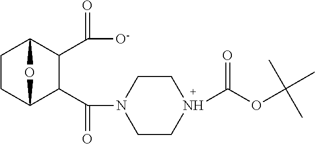 |
| Compound 102E | 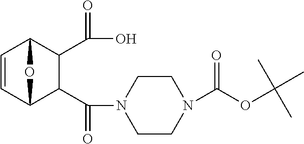 |
| Compound 103 | 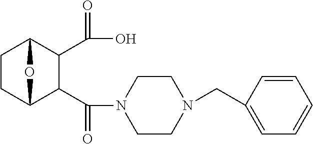 |
| Compound 103E | 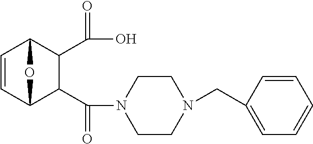 |
| Compound 104 | 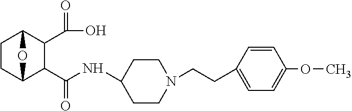 |
| Compound 104E | 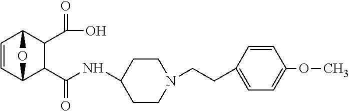 |
| Compound 105 | 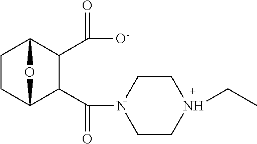 |
| Compound 105E | 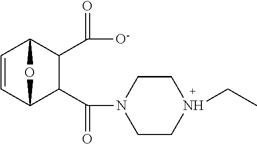 |
| Compound 106 | 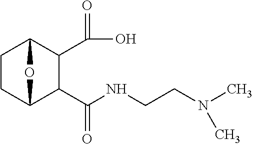 |
| Compound 106E | 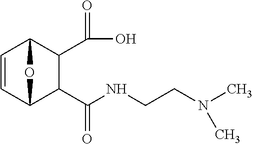 |
| Compound 107 | 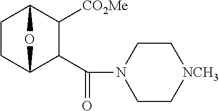 |
| Compound 107E | 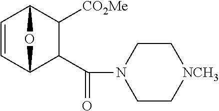 |
| Compound 108 | 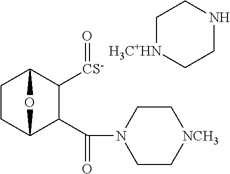 |
| Compound 108E | 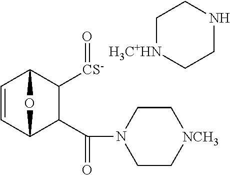 |
| Compound 109 | 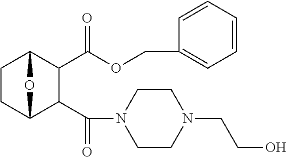 |
| Compound 109E | 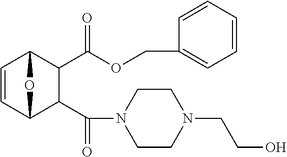 |
| Compound 110 | 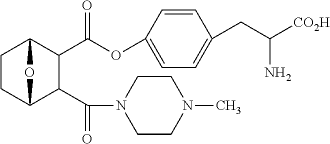 |
| Compound 110E | 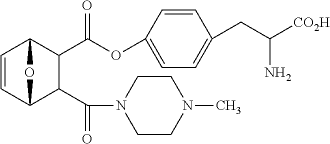 |
| Compound 111 | 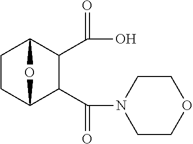 |
| Compound 112 | 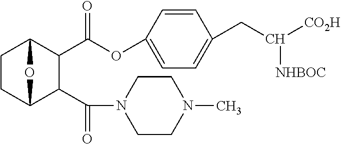 |
| Compound 112E | 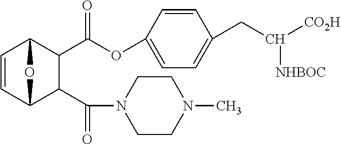 |
| Compound 113 | 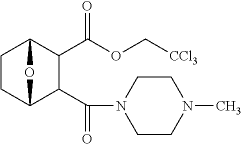 |
| Compound 113E | 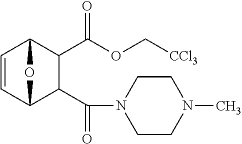 |
| Compound 114 | 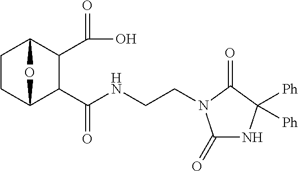 |
| Compound 114E | 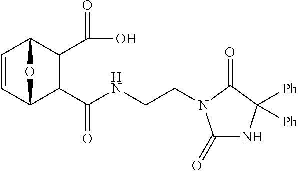 |

### Experimental Details

### Example 1

### Inhibition of PP2A Diminishes a Major Defense Against DNA Damage, Cell-Cycle Arrest by p53 by Reducing the Cell Concentration of p53

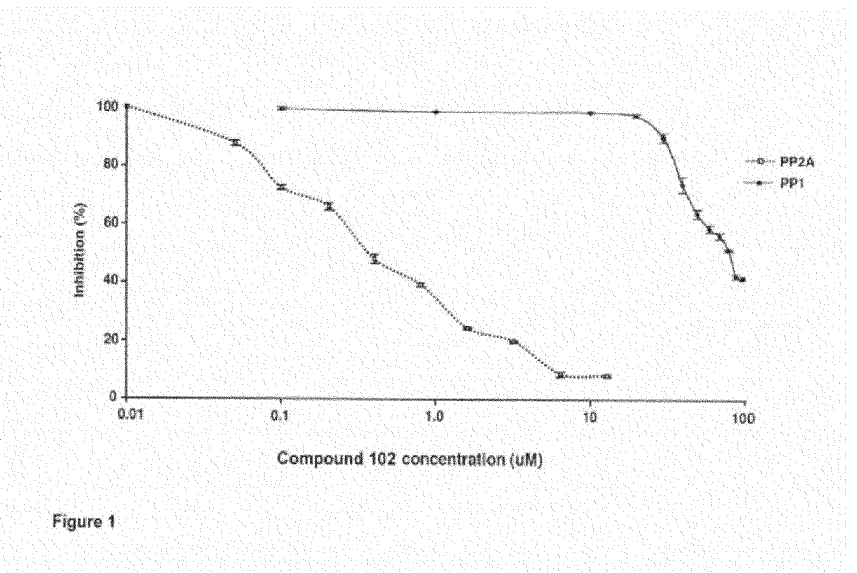

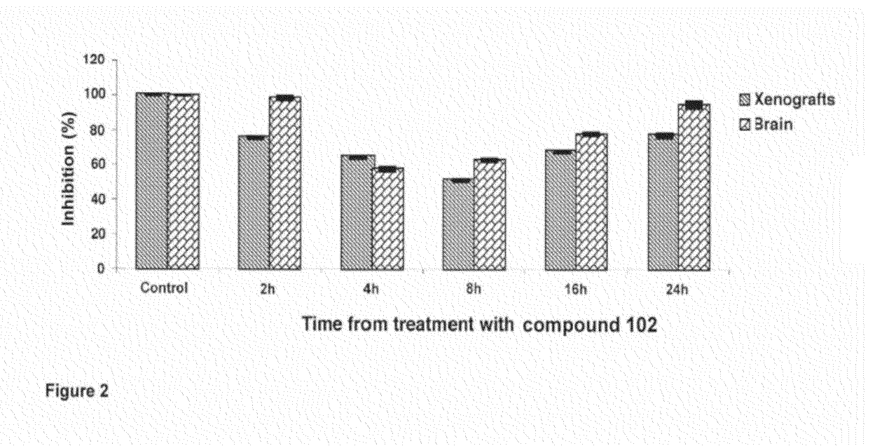

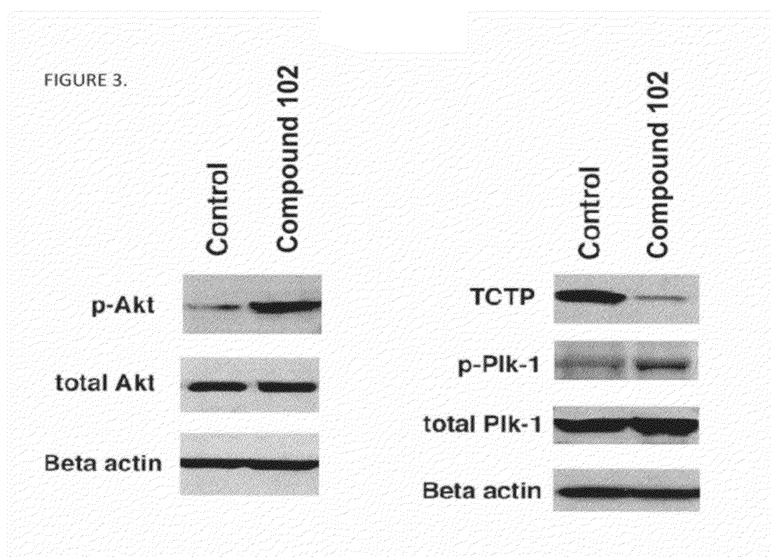

Compound 102 inhibits PP2A and PP1 in lysates of human glioblastoma cell line U87 (FIG. 1) and inhibits PP2A in vivo in xenografts of U87 (subcutaneous) and in normal brain tissue of SCID mice (FIG. 2). Exposure of U87MG cells in culture to compound 102 resulted in increased phosphorylated Akt (pAkt-1), Plk-1 (pPlk-1), and a marked decrease in translationally controlled tumor protein (TCTP; FIG. 3). TCTP is an abundant, highly conserved, multifunctional protein that binds to and stabilizes microtubules before and after mitosis and also exerts potent anti-apoptotic activity (Bommer and Thiele, 2004; Yarm, 2002; Susini et al, 2008). Decreasing TCTP with anti-sense TCTP has been shown by others to enhance tumor reversion of v-src-transformed NIH 3T3 cells and reduction of TCTP is suggested to be the mechanism by which high concentrations of certain anti-histaminics and psychoactive drugs inhibit growth of a human lymphoma cell line (Tuynder et al, 2004).

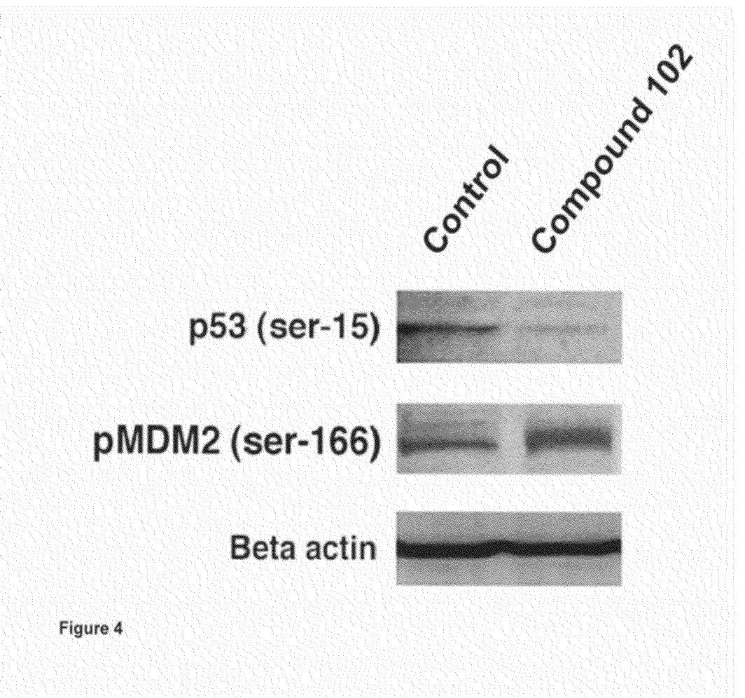

pAkt-1 phosphorylation at Ser308 indicates downstream activation of the phosphatidylinositol-3-kinase (PI3K) pathway, an event generally considered to be growth-promoting (Brazil et al, 2004). Akt-1 activation, however, may be anti- or proapoptotic depending on the context of cell signaling (Andrabi et al, 2007). Compound 102 inhibition of PP2A increased pAkt-1 and activated Plk-1, a regulator of a mitotic checkpoint and of the activity of TCTP. Compound 102 exposure also increased phosphorylated MDM2, the primary regulator of p53 activity (Vogelstein et al, 2000; Vazquez et al, 2008) and decreased the abundance of p53 (FIG. 4). pAkt-1 can directly phosphorylate MDM2, increasing its stability, and can phosphorylate MDMX, which binds to and further stabilizes MDM2 (Olivier et al, 2008). Thus inhibition of PP2A diminishes a major defense against DNA damage, cell-cycle arrest by p53.

### Example 2

### Effects of Compound 102 on Decreasing p53 are Maintained in the Face of DNA Damage by the DNA-Damaging Agent, Temozolomide (TMZ)

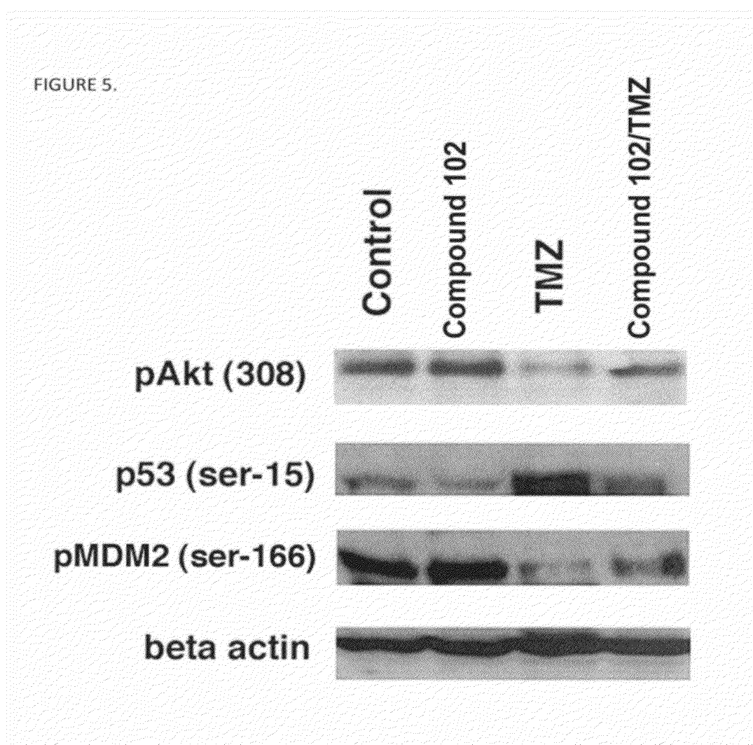

We found that there is an increase in tumor cell killing by compound 102 plus TMZ because inhibition of PP2A renders cells more vulnerable to TMZ by inhibiting p53 mediated DNA damage arrest (Lu et al., 2009). The effects of compound 102, TMZ, and compound 102 plus TMZ on the amount of pAkt, p53 and MDM2 in U87MG, a cell line with wild-type p53 (Short et al, 2007) were assessed by Western blots. Exposure of U87MG cells to compound 102 alone for 24 hours increased both pAkt-1 and MDM2 and eliminated p53; TMZ alone decreased pAkt-1, increased p53, and had little effect on MDM2. Adding compound 102 prevented the decrease in pAkt-1 caused by TMZ alone and increased MDM2 in the face of continued increased expression of p53 (FIG. 5).

### Example 3

### Effects of PP2A Inhibition by Compound 102 on p53 are Mimicked by Another Known Inhibitor of PP2A, Okadaic Acid

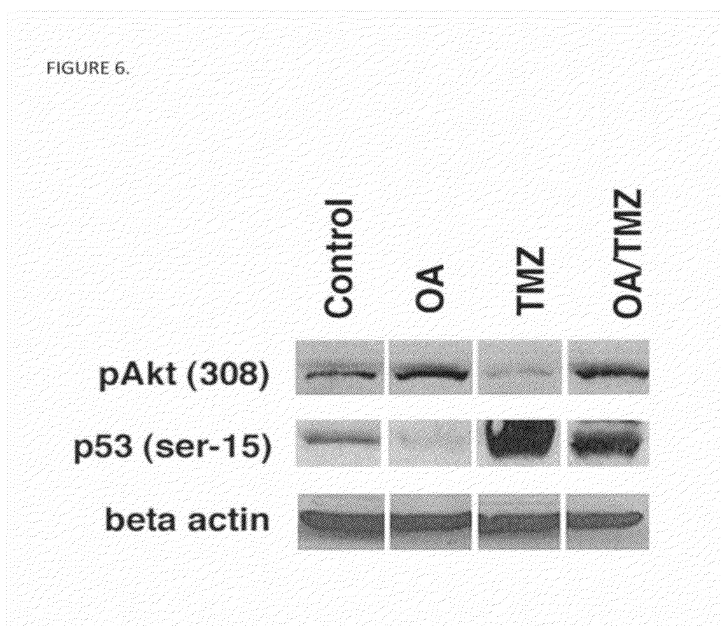

The same molecular changes in pAkt-1 and p53 induced by compound 102, TMZ, and compound 102 plus TMZ occurred in U87 cells when Okadaic acid, at a concentration (2 nM) that is expected to inhibit PP2A and not PP1 (Hart et al, 2004), was substituted for compound 102 (FIG. 6) supporting the hypothesis that the effects of compound 102 result from inhibition of PP2A.

### Example 4

Recently, several research teams have discovered that silencing the activity of the small P53 pathway increases the success rate of re-programming. In 2009, five groups reported that inhibiting p53 activity greatly improved the efficiency of iPS generation (Hong et al, 2009; Li et al, 2009; Kawamura et al, 2009; Marion et al, 2009; Utikal et al, 2009). These groups have used genetic and molecular techniques to reduce or eliminate p53 function and showed that this manipulation was associated with increased re-programming efficiency of human somatic cells.

Although the inhibition of p53 activity has generated considerable excitement because of the marked improvement in the generation of iPS, there is concern that elimination of p53 function, an activity which is a major defense mechanism for protecting the cell from replicating damaged DNA, could not be used for inducing iPS cells for clinical use (Krizhanovsky and Lowe, 2009).

A series of novel PP2A inhibitors, compounds 100-114 and compounds 100E-110E and 112E-114E have been developed. We have shown that Compound 102 inhibits PP2A approximately 200 times as efficiently as PP1 (Lu et al, 2009). A consequence of inhibiting PP2A with Compound 102 is a marked reduction in the abundance of p53, even in the presence of a DNA damaging agent such as temomozolomide or doxorubicin. Okadaic acid, a known inhibitor of PP2A at nanomolar concentrations, mimics the effects of Compound 102 on reducing p53 activity (Lu et al, 2009).

Compound 102 and its water soluble analog Compound 100, can be administered to animals (mice and rats) at doses which inhibit PP2A in subcutaneous xenografts of human cancers and in the normal brain tissue of these animals (FIG. 2). Inhibition of PP2A after a single intraperitoneal injection achieves maximum inhibition of PP2A in tissue at 6-8 hours, after which PP2A recovers. Inhibition of PP2A is associated with a marked diminution of p53 in tissue, mediated apparently by marked induction of phosphorylated MDM2 (p-MDM2). MDM2 is the prime regulator of p53 abundance.

Thus, the small molecule Compound 100 and associated homologs, for example the compounds disclosed herein at Table 1, can be used to reduce p53 activity by inhibition of PP2A providing a window of opportunity for re-programming somatic cells into iPS cells with subsequent return of p53 activity once the PP2A inhibitor is withdrawn, thereby increasing the efficiency of the process for producing iPS cells.

### REFERENCES

1. News Focus (2008) “A Seismic Shift For Stem Cell Research”, Science, 319:560-563.
2. Berge et al. (1977) “Pharmaceutical Salts”, J. Pharm. Sci. 66:1-19
3. Bommer, U A, and Thiele, B J (2004), “The translationally controlled tumor protein (TCTP.” International Journal of Biochemistry & Cell Biology, Vol 36 pp. 379-385.
4. Brazil, D. P., Yang, Z.-Z. & Hemmings, B. Advances in protein kinase B signalling: AKTion on multiple fronts. Trends in Biochemical Sciences 29, 233-242 (2004).
5. Hart, M. E., Chamberlin, A. A., Walkom, C., Sakoff, J. A. & McCluskey, A. Modified nor-cantharidins: synthesis, protein phosphatases 1 and 2A inhibition, and anti-cancer activity. Bioorg. Med. Chem. Letters. 14, 1969-1973 (2004).
6. Hong et al. (2009) “Suppression Of Induced Pluripotent Stem Cell Generation By The p53-p21 Pathwway”, Nature, 460:1132-1135.
7. Kawamura, T. et al. (2009) “Linking The p53 Tumour Suppressor Pathway To Somatic Cell Reporgramming”, Nature 460:1140-1144.
8. Krizhanovsky, K. and Lowe, S. W. (2009) “The Promises And Perils Of p53”, Nature 460:1085-1086.
9. Li, H. et al. (2009) “The Ink4/Arf Locus Is A Barrier for iPS Cell Reprogramming” Nature 460(1136-1139).
10. Lu, J. et al. (2009) “Inhibiting Of Serine/Threonine Phosphatase PP2A enhances Cancer Chemotherapy By Blocking DNA Damage Induced Defense Mechanisms”, PNAS, 106(28):11697-11702.
11. Marion, R. M. et al. (2009) “A p53-Mediated DNA Damage Response Limits Reprogramming To Ensure iPS Cell Genomic Integrity”, Nature 460:1149-1153.
12. Okita, K et al. (2007) “Generation Of Germline-Competent Induced Pluripotent Stem Cells”, Nature 448(7151):260-262.
13. Olivier, M. et al. Recent advances in p53 research: an interdisciplinary perspective. Cancer Gene Therapy 19 Sep. 2008; doi:10.1038/cgt.2008.69, pages 1-12 (2008).
14. Short, S. C. et al. DNA repair after irradiation in glioma cells and normal human astrocytes. Neuro-Oncology 9, 404-411 (2007).
15. Susini, L et al. (2008). “TCTP protects from apoptotic cell death by antagonizing bax function.” Cell Death and Differentiation, 15 Feb. 2008:DOI:10.1038/cdd.2008.18.
16. Takahashi, K and Yamanaka, S. (2006) “Induction Of Pluripotent Stem Cells From Mouse Embryonic And Adult Fibroblast Cultures By Defined Factors”, Cell, 126(4):652-655.
17. Utikal, J. et al. (2009) “Immortalization eliminates A Roadblock During Cellular Reprogramming Into iPS Cells”, Nature, 460: 1145-1148.
18. Vazquez, A., Bond, E E, Levine, A. J. & Bond, G. L. The genetics of the p53 pathway, apoptosis and cancer therapy. Nature Rev. Cancer 7, 979-987 (2008).
19. Vogelstein, B., Lane D. & Levine, A. J. Surfing the p53 network. Nature 408, 307-310 (2000).
20. Wernig, M. (2007) “In Vitro Reprogramming Of Fibroblasts into a pluripotent ES-Cell-Like State”, Nature 448(7151):318-324.
21. Yarm, F R, (2002). “Plk Phosphorylation Regulates the Microtubule-Stabilizing Protein TCTP.” Molecular and Cellular Biology, Vol. 22, pp. 6209-6621.
22. Tuynder, M. et al. (2004). “Translationally controlled tumor protein is a target of tumor reversion.” PNAS, Vol. 101, pp. 15364-15369.
23. U.S. Patent Application Publication NO. 2006/0205075 published Sep. 14, 2006.
24. U.S. Patent Application Publication No. 2009/0047263, published Feb. 19, 2009.
25. U.S. Patent Application Publication No. 2009/0227032 published Sep. 10, 2009.
26. U.S. Patent Application Publication No. 2009/0246875 published Oct. 1, 2009.

1. A method of inhibiting the function of p53 in a cell comprising contacting the cell with an effective amount of a PP2A inhibitor, wherein the PP2A inhibitor is a compound having the structure: 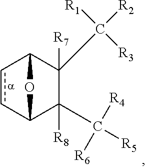
wherein
bond α is present or absent;
R₁ and R₂ is each independently H, O⁻ or OR₉,
where R₉ is H, alkyl, substituted alkyl, alkenyl, alkynyl or aryl,
or R₁ and R₂ together are ═O;
R₃ and R₄ are each different, and each is OH, O⁻, OR₉, OR₁₃, O(CH₂)₁₋₆R₉, SH, S⁻, SR₉, 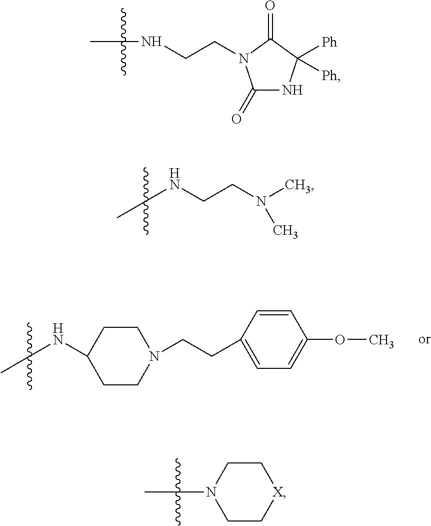
where X is O, S, NR₁₁, or N⁺R₁₁R₁₁,
where each R₁₁ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O, 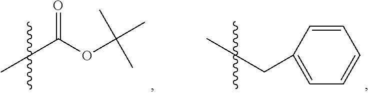
—CH₂CN, —CH₂CO₂R₁₂, —CH₂COR₁₂, —NHR₁₂ or —NH⁺(R₁₂)₂, where each H₁₂ is independently alkyl, alkenyl or alkynyl, each of which is substituted or unsubstituted, or H;
R₁₀ is substituted alkyl, substituted alkenyl, substituted alkynyl, or substituted aryl;
R₅ and R₆ is each independently H, OH, or R₅ and R₆ taken together are ═O; and
R₇ and R₈ is each independently H, F, Cl, Br, SO₂Ph, CO₂CH₃, or SR₁₃,
where R₁₃ is H, aryl or a substituted or unsubstituted alkyl, alkenyl or alkynyl,
or a salt, enantiomer or zwitterion of the compound,
so as to thereby inhibit the function of p53 in the cell.

2. The method of claim 1, wherein the compound has the structure 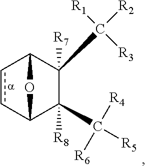
or a salt, enantiomer or zwitterion of the compound.

3. The method of claim 2, wherein the compound has the structure 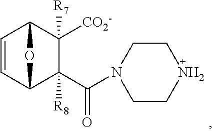
or a salt, enantiomer or zwitterion of the compound.

4. The method of claim 1, wherein R₄ is 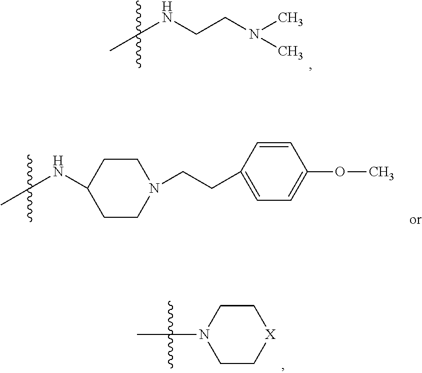
where X is O, NR₁₁, or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro when R₁ and R₂ are ═O, 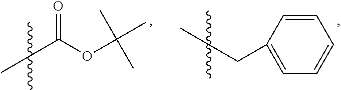
—CH₂CN, —CH₂CO₂R₁₂, —CH₂COR₁₂, —NHR₁₂ or —NH⁺(Rᵤ)₂, where R₁₂ is H or alkyl,
or a salt, enantiomer or zwitterion of the compound.

5. The method of claim 4, wherein the compound is 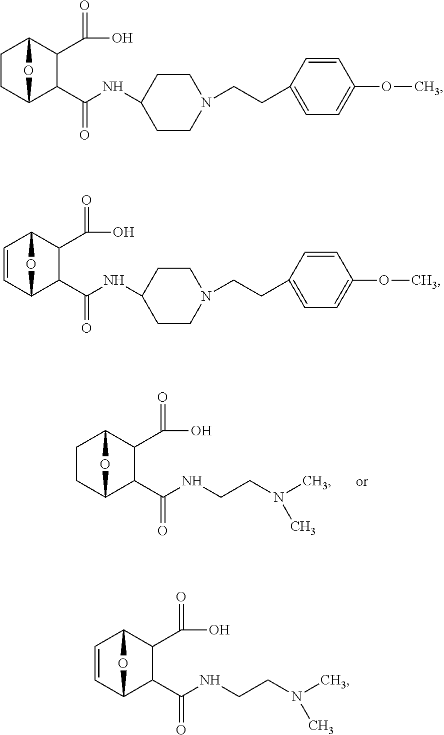
or a salt, enantiomer or zwitterion of the compound.

6. The method of claim 1, wherein the compound has the structure 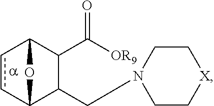
wherein bond α is present or absent; R₉ is present or absent and when present is H, C₁-C₁₀ alkyl, C₂-C₁₀ alkenyl or phenyl; and X is O, S, NR₁₁ or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro, 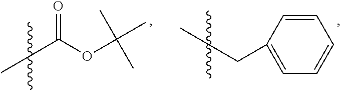
—CH₂CO₂R₁₂, —CH₂COR₁₂, —CH₂CN, or —CH₂CH₂R₁₆, where R₁₂ is H or alkyl, and where R₁₆ is any substituent that is a precursor to an aziridinyl intermediate,
or a salt, zwitterion or enantiomer of the compound.

7. The method of claim 6, wherein the compound has the structure 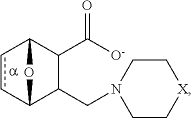
wherein,
bond α is present or absent;
X is O, S, NR₁₁ or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro, 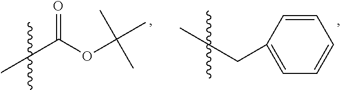
—CH₂CO₂R₂, —CH₂COR₁₂, —CH₂CN, or —CH₂CH₂R₁₆, where R₁₂ is H or alkyl, and where R₁₆ is any substitutent that is a aziridinyl intermediate,
or a salt, zwitterion or enantiomer of a compound.

8. The method of claim 1, wherein the compound has the structure 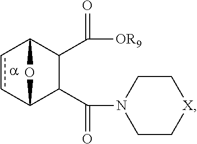
wherein
bond α is present or absent;
R₉ is present or absent and when present is H, alkyl, alkenyl, alkynyl or phenyl; and
X is O, NR₁₁, or N⁺R₁₁R₁₁,
where each R₁₁ is independently H, alkyl, substituted C₂-C₁₂ alkyl, alkenyl, substituted C₄-C₁₂ alkenyl, alkynyl, substituted alkynyl, aryl, substituted aryl where the substituent is other than chloro, 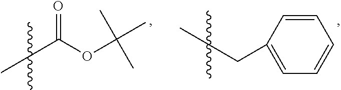
—CH₂CN, —CH₂CO₂R₁₂, or —CH₂COR₁₂, where R₁₂ is H or alkyl,
or a salt, zwitterion, or enantiomer of the compound.

9. The method of claim 8, wherein the compound has the structure 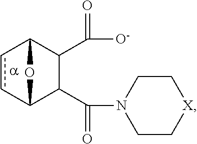
wherein
bond α is present or absent;
X is O or NH⁺R₁₁,
where R₁₁ is H, alkyl, substituted C₂-C₁₂ alkyl, alkenyl, substituted C₄-C₁₂ alkenyl, alkynyl, substituted alkynyl, aryl, substituted aryl where the substituent is other than chloro, 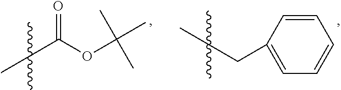
—CH₂CN, —CH₂CO₂R₂₂, or —CH₂COR₁₂, where R₁₂ is H or alkyl,
or a salt, enantiomer or zwitterion of the compound.

10. (canceled)

11. The method of claim 8, wherein bond α is absent.

12. The method of claim 11, wherein the compound has the structure: 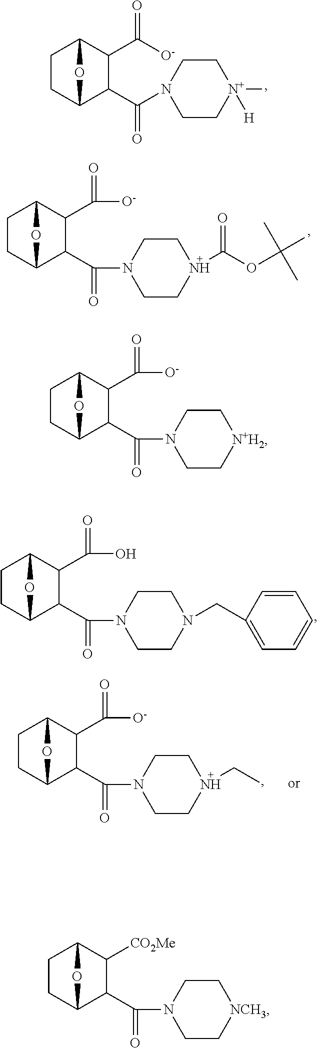
or a salt, enantiomer or zwitterion of the compound.

13. The method of claim 8, wherein bond α is present and wherein the compound has the structure 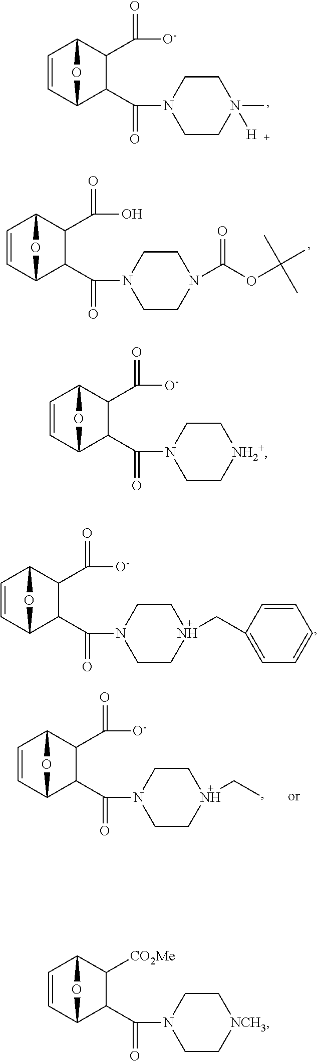
or a salt, enantiomer or zwitterion of the compound.

14. The method of claim 8, wherein the compound has the structure 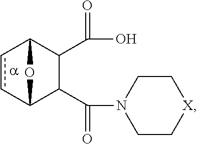
wherein
bond α is present or absent; X is NH⁺R₁₁,
where R₁₁ is present or absent and when present R₁₁ is alkyl, substituted C₂-C₁₂ alkyl, alkenyl, substituted C₄-C₁₂ alkenyl, 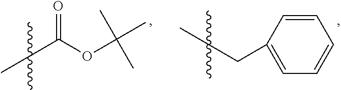
—CH₂CN, —CH₂CO₂R₁₂, or —CH₂COR₁₂, where R₁₂ is H or alkyl,
or a salt, enantiomer or zwitterion of the compound

15. The method of claim 1, wherein the compound has the structure

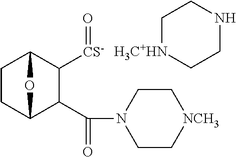

16. The method of claim 1, wherein
R₃ is OR₁₀ or O(CH₂)₁₋₆R₉,
where R₉ is aryl or substituted ethyl;
where R₁₀ is substituted phenyl, wherein the substituent is in the para position;
R₄ is 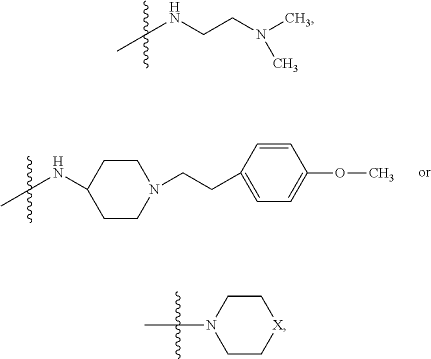
where X is O, S, NR₁₁, or N⁺R₁₁R₁₁,
where each Rᵤ is independently H, alkyl, hydroxyalkyl, substituted C₂-C₁₂ alkyl, alkenyl, substituted C₄-C₁₂ alkenyl, alkynyl, substituted alkynyl, aryl, substituted aryl where the substituent is other than chloro, 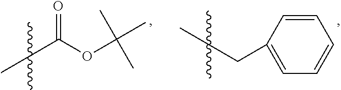
—CH₂CN, —CH₂CO₂R₁₂, —CH₂COR₁₂, —NHR₁₂ or —NH⁺(R₁₂)₂, where R₁₂ is alkyl, alkenyl or alkynyl, each of which is substituted or unsubstituted, or H;
or where R₃ is OH and R₄ is 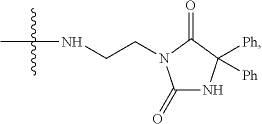
or a salt, enantiomer or zwitterion of the compound.

17. (canceled)

18. The method of claim 16, wherein the compound has the structure 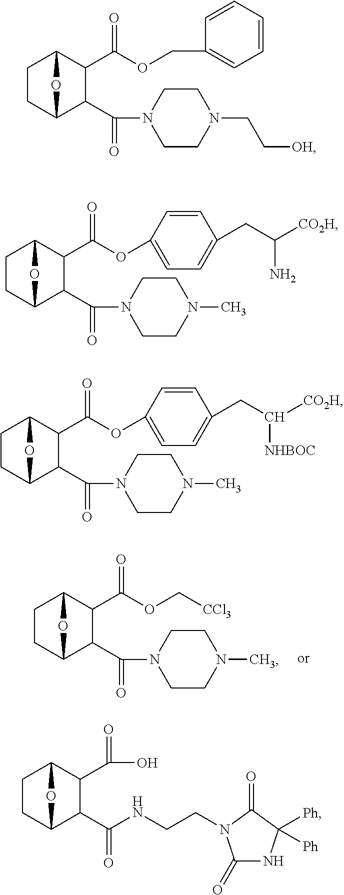
or a salt, enantiomer or zwitterion of the compound.

19. A process for producing, or increasing the likelihood of producing, an induced pluripotent stem (iPS) cell comprising contacting a somatic cell expressing at least one gene that encodes a reprogramming factor with an amount of a PP2A inhibitor effective to produce the iPS cell, or increase the likelihood of producing an iPS cell.

20. A process for reversibly inhibiting p53 function during production of an induced pluripotent stem (iPS) cell from a somatic cell expressing at least one gene that encodes a reprogramming factor comprising contacting the somatic cell with an amount of a PP2A inhibitor effective to reversibly inhibit p53 function.

21. (canceled)

22. The process of claim 19 for increasing the production efficiency of induced pluripotent stem (iPS) cells, wherein the process for production of the iPS cells comprises transforming a population of somatic cells with at least one gene that encodes a reprogramming factor, and contacting the population of somatic cells with an amount of a PP2A inhibitor effective to increase the efficiency of the production of iPS cells.

23. The process of claim 20 comprising transforming a somatic cell to express at least one gene that encodes a reprogramming factor such that the somatic becomes an iPS, and contacting the somatic cell with an amount of a PP2A inhibitor effective to transiently reduce the function of p53 in the cell.

24-41. (canceled)
